(19) United States
(12) Patent Application Publication
(10) Pub. No.: US 2012/0214230 A1
(43) Pub. Date: Aug. 23, 2012

(54) CELL CULTURE/HANDLING PRODUCT AND METHOD FOR PRODUCTION AND USE THEREOF

(75) Inventors: Cecilia Anneren, Uppsala (SE); Gunnar Glad, Uppsala (SE); Christine Sund Lundstrom, Uppsala (SE)

(73) Assignee: GE HEALTHCARE BIO-SCIENCES AB, UPPSALA (SE)

(21) Appl. No.: 13/503,388

(22) PCT Filed: Oct. 21, 2010
(86) PCT No.: PCT/SE2010/051138

§ 371 Date: Apr. 23, 2012

(30) Foreign Application Priority Data
Oct. 22, 2009 (SE) 0950778-1

(51) Int. Cl. C12N 5/071; C09D 105/02; C12M 3/00

(52) U.S. Cl. 435/325; 435/304.1; 435/305.1; 435/289.1; 435/303.1; 106/162.1

(57) ABSTRACT

The present invention relates to a fast a simple coating procedure for coating of cell culture and/or handling surfaces to prevent cell adhesion and growth. The invention relates to a cell culture/handling product coated with phenyl dextran, as well as methods of producing and using it.

### FIELD OF THE INVENTION

The present invention is within the cell biology field. More closely it relates to a cell culture and/or handling product and a method of producing said cell culture/handling product using phenyl dextran for coating thereof. The invention also relates to applications of said cell culture/handling product.

### BACKGROUND OF THE INVENTION

For cell culture, handling, and storage, there is a high demand for materials that efficiently inhibit cell attachment or adherence. Non-specific sticking or attachment to plastics on tissue culture plates/flasks, tubings, or bags may be a major problem when expanding, handling, transferring or storing cells. For instance, when culturing adherent cells on microcarriers some cells may adhere to the bottom of the compartment during seeding, or the microcarriers may stick to the cell culture bag once they become confluent with cells. Moreover, many non-treated tissue culture plastics, intended for suspension cultures allows for attachment of adherent cells, such as mesenchymal stem cells, which is a problem if attachment needs to be avoided.

It is known that polyethylene glycol (PEG) and dextran have cell adhesion resistive properties. For example:

1) Monchaux, E., and Vermette, P. (2008). Cell adhesion resistance mechanisms using arrays of dextran-derivative layers. J Biomed Mater Res A 85, 1052-1063. This work describes the use of carboxy methyl dextran to coat surfaces. Carboxy methyl dextrans (CMDs) were produced by reacting bromoacetic acid with dextrans for 16 h, followed by dialyses (3×24 h) and lyophilization. Borosilicate glass was acid washed in a overnight procedure, followed by surface-modification with n-heptylamine in a plasma polymerization reactor. CMD solutions were activated with EDC and NHS, and dispensed on the surface—modificated surface, followed by an overnight coupling reaction plus washings for 24 h. The whole procedure needs over 1 week to perform.

2) Massia, S. P., Stark, J., and Letbetter, D. S. (2000). Surface-immobilized dextran limits cell adhesion and spreading. Biomaterials 21, 2253-2261. This paper describes the use of immobilized dextran to prevent cell adhesion and spreading. Dextran was oxidized in a 24 h reaction with sodium periodate, followed by dialyses and lyophilization. PET coverslips and glass micro-slides were cleaned in 6-step procedure. PET coverslips were then surface-modified with ethylene diamine, and glass micro-slides were treated with 3-aminopropyltriethoxysilane. Amine modified surfaces were finally immersed for 16 h in oxidized dextran solutions. After decanting and incubation for 2 h in a sodium borohydride solution, the dextran coated surfaces were rinsed and dried. The whole procedure needs approx. 1 week to perform.

Thus, the prior art methods are very complicated and time consuming.

### SUMMARY OF THE INVENTION

The present invention provides a simple and convenient procedure to obtain low-adherent cell culture and handling products at a low cost.

The present inventors have discovered that phenyl dextran can be used as a surface coating to reduce cell attachment to hydrophobic surfaces such as cell culture plastic. Moreover, the inventors have found that the coating procedure is quick and easy and that an extremely low concentration of phenyl dextran can be used to achieve the desired function. The coated product may be used for culture or handling of any cell type or tissue without affecting their inherent properties.

The invention is also useful when growing cells on microcarriers or scaffolds to avoid non-specific attachment to the bottom of the cell culture compartment.

In a first aspect the invention relates to an apparatus for culturing and/or handling cells, said apparatus having at least one surface which is exposed to said cells, wherein said at least one surface is coated with phenyl dextran. The apparatus may comprise a cell culture and/or handling product which may be any container or vessel suitable for cell culture, handling, transfer or storage of cells. Preferably the inner surface of such a vessel or container is a hydrophobic surface. Alternatively, a non-hydrophobic vessel or container is used, which is provided with a hydrophobic surface before the phenyl dextran is added.

The cell culture and/or handling product may for example be a culture plate, culture flask, microtiter plate, tubing, beaker or bag, wherein at least one inner surface thereof is coated with phenyl dextran.

In a second aspect, the invention relates to a method of producing a cell culture/handling product, comprising coating a phenyl dextran solution in a concentration of 0.1-5 mg/ml on a hydrophobic surface without pre-treatment of said surface.

In a third aspect, the invention relates to a method to prevent cell adhesion or attachment of adherent cells to surfaces of cell culture/handling products, comprising culture of cells in a cell culture and/or handling product as described above.

Preferably, the cells are adherent stem cells, primary cells, or cell lines, pieces of tissue/organs or suspension cells, kept as single cells, three dimensional structures such as spheres or adhered to a secondary structure such as carriers, scaffolds or discs.

In a further embodiment, the invention relates to a method comprising addition of microcarriers for the cells to grow on. The cells will grow on the microcarriers and not adhere to the surface of the cell culture product.

In a third aspect, the invention relates to use of phenyl dextran to coat a cell culture/ handling surface to prevent cell adhesion. Preferably, the phenyl dextran is coated in a concentration of 0.1-5 mg/ml.

In a fourth aspect, the invention relates to a kit comprising a solution of phenyldextran and cell culture/handling containers as well as instructions how to coat at least one inner surface of the containers with the solution to prevent cell adhesion on said surface(s). Preferably, the phenyl dextran solution is in a concentration of 0.1-5 mg/ml.

### BRIEF DESCRIPTION OF THE DRAWINGS

FIG. 1 Schematic representation of phenyl dextran coating on a culture plate and addition of cells (MSCs) to the culture plate; and

FIG. 2 shows mesenchymal stem cells cultured on a non-coated surface and a surface coated according to the invention

### DETAILED DESCRIPTION OF THE INVENTION

### Materials and Methods

Microtiter Plates:

Non-treated polystyrene microtiter plates from Nunc were coated with three different concentrations of phenyl dextran: 10 mg/ml, 1 mg/ml and 0,1 mg/ml. Two commercially available culture plates with ultra low attachment were used for comparison: 1) Nunc MPC treated, order number: 145383 and 2) Costar Ultra low attachment plates (order number: 3473).

Phenyl Dextran:

Phenyl dextran (Dextran T40, GE Healthcare Biosciences AB) with an average molecular weight of 40000 g/mol substituted with phenyl glycid ether.

Microcarriers:

Cytodex 1 and cytodex 3 (GE Healthcare Biosciences AB).

Cells:

Human mesenchymal stem cells (hMSC) were used for plating at 20 000 cells/well when testing with microcarriers and 40 000 cells/well when testing attachment to plates.

### EXPERIMENTAL PART

### Coating of Microtiter Plates

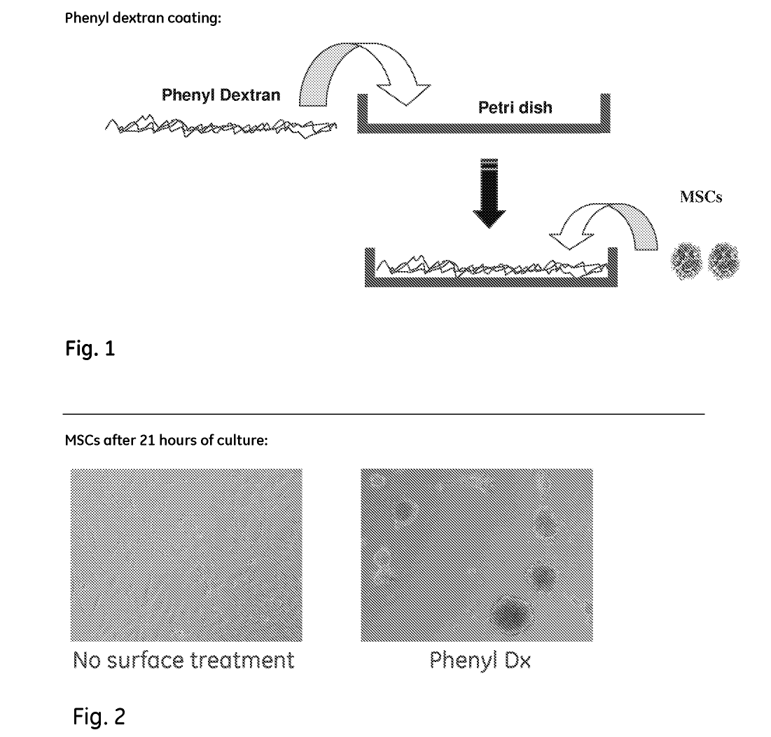

Non-treated polystyrene microtiter plates from Nunc were coated with three different concentrations of phenyldextran, 10 mg/ml, 1 mg/ml and 0,1 mg/ml. The general procedure is outlined in FIG. 1.

Coating Procedure:

Add 500 μl of phenyl dextran solution, leave on plates for 15 minutes

Discard solution

Add 1000 μl of phosphate buffer solution (PBS)×10 minutes×3 times

For cell culture, 500 μl of basal cell culture media is added and left for 10 minutes. Thereafter, complete media and cells are added.

The phenyl groups on the dextran adsorb to hydrophobic surfaces, which will make the surface more hydrophilic. The coating procedure is simple and not time consuming.

### Cell Culture

Mesenchymal stem cells (MSCs) at 40 000 cells/well were added to the plates coated according to the invention and the comparison plates. After a 21 hour cultivation period, cell attachment was assessed. No attachment of cells to the tissue culture plastic was observed for the phenyl dextran coated plates (all concentrations worked equally well) and the Costar plates. However, the Nunc plates exhibited an uneven coating with cells attaching to parts of the plates.

When cells were seeded on Phenyl dextran-coated plastic, they did not attach at all. Instead they formed free-floating spherical cell clusters (see FIG. 2).

### Adding Microcarriers to the Cell Culture

When adding Cytodex microcarriers to the Costar and phenyl dextran coated plates, all cells (20 000 cells/well) attached to the microcarriers and no cells attached to the bottom of the wells of the microtiter plate. The cells grew well on the carriers and no difference in cell morphology was observed when cultured on microcarriers in the phenyl dextran coated plates, indicating that the phenyl dextran is not leaking to also coat the carriers or is toxic to the cells.

### Results

The result was that the phenyl dextran coated plates were better than Nunc MPC treated and equal to Costar Ultra low attachment plates, when assessing attachment but at a much lower cost.

The present results indicate that phenyl dextran can completely inhibit cell attachment without altering the proliferation, survival or multi-potency of cells.

The coating procedure is a simple, fast and flexible and could be applied to other types of hydrophobic materials to achieve ultra-low cell attachment properties.

### Application Examples of the Invention

There are many occasions when attachment of cells/tissue to a culture dish, tubing, bag or other material should be avoided such or for example: 1) When growing cells as free-floating spheres such as embryoid bodies or neurospheres ; 2) When culturing pieces of tissue/organs in vitro that need to maintain their three-dimensional structure; 3) When growing cells on another structure in a tissue culture plate, flask or bag, such as carrier, scaffold or other three-dimensional biomaterial 4) when culturing suspension cells or cells growing on carriers in spinner flasks or bags (e.g. the WAVE Bioreactor™) 5) when transferring sticky cells through at tubing or other device 6) when storing or working with cells to avoid unspecific sticking to the inside of the container.

1. An apparatus for culturing and/or handling cells, comprising at least one surface which is exposed to said cells, wherein the at least one surface is coated with phenyl dextran.

2. The apparatus of claim 1, wherein said at least one surface is a hydrophobic surface.

3. The apparatus of claim 1, wherein said apparatus is a cell culture product, cell incubator, cell handling device, such as a culture plate, culture flask, microtiter plate, tubing, beaker or bag, and wherein at least one inner surface thereof is coated with phenyl dextran.

4. A method of producing a cell culture/handling product, comprising coating a phenyl dextran solution in a concentration of 0.1-5 mg/ml on a hydrophobic surface.

5. A method to prevent cell adhesion or attachment of adherent cells to surfaces of cell culture/handling products, comprising culture of cells in the apparatus for culturing and/or handling the cells of claim 1.

6. The method of claim 5, wherein the cells are adherent stem cells, primary cells, or cell lines, pieces of tissue/organs or suspension cells, kept as single cells, three dimensional structures such as spheres or adhered to a secondary structure such as carriers, scaffolds or discs.

7. The method of claim 5, comprising an addition of microcarriers for the cells to grow on.

8-9. (canceled)

10. A kit comprising a solution of phenyldextran and cell culture/handling containers as well as instructions how to coat at least one inner surface of the containers with the solution to prevent cell adhesion on said surface(s).

11. The kit of claim 10, where the phenyl dextran solution is in a concentration of 0.1-5 mg/ml.
